(19) United States
(12) Patent Application Publication
(10) Pub. No.: US 2023/0201805 A1
(43) Pub. Date: Jun. 29, 2023

(54) DEHYDROGENATION CATALYST COMPOSITION

(71) Applicant: UOP LLC, Des Plaines, IL (US)

(72) Inventors: Phuong T.M. Do, Mount Prospect, IL (US); Manuela Serban, Northbrook, IL (US); J.W.Adriaan Sachtler, Des Plaines, IL (US); Matthew C. Cole, Evanston, IL (US)

(21) Appl. No.: 18/081,345

(22) Filed: Dec. 14, 2022

Related U.S. Application Data
(62) Division of application No. 16/548,879, filed on Aug. 23, 2019.

(51) Int. Cl. B01J 23/58; C07C 5/32; B01J 23/62; B01J 35/10; B01J 21/04; C01F 7/02

(52) U.S. Cl. CPC B01J 23/58; C07C 5/325; B01J 23/62; B01J 35/1019; B01J 21/04; C01F 7/02; B01J 23/626; C07C 5/42

(57) ABSTRACT

A catalytic composite comprises a first component selected from Group VIII noble metal components and mixtures thereof, a second component selected from one or more of alkali and alkaline earth metal components, and a third component selected from one or more of tin, germanium, lead, indium, gallium, and thallium, all supported on an alumina support comprising delta alumina having an X-ray diffraction pattern comprising at least three 2θ diffraction angle peaks between 32.0° and 70.0°. The at least three 2θ diffraction angle peaks comprise a first 2θ diffraction angle peak of 32.7°±0.4°, a second 2θ diffraction angle peak of 50.8°±0.4°, and a third 2θ diffraction angle peak of 66.7°±0.8°, wherein the second 2θ diffraction angle peak has an intensity of less than about 0.06 times the intensity of the third 2θ diffraction angle peak.

This is a divisional application to application Ser. No. 16/548,849, filed Aug. 23, 2019 which is incorporated herein in its entirety. The field relates to a catalytic composite. Particularly, the field relates to a catalytic composite comprising an alumina support.

### FIELD

### Background

Petroleum refining and petrochemical processes frequently involve the selective conversion of hydrocarbons with a catalyst. The dehydrogenation of hydrocarbons is an important commercial process because of the great demand for dehydrogenated hydrocarbons for the manufacture of various chemical products such as detergents, high octane gasolines, pharmaceutical products, plastics, synthetic rubbers, and other products well known to those skilled in the art. One example of this process is dehydrogenating isobutane to produce isobutylene which can be polymerized to provide tackifying agents for adhesives, viscosity-index additives for motor oils, impact-resistant and anti-oxidant additives for plastics and a component for oligomerized gasoline. Another example is dehydrogenation of a propane rich feedstock to produce propylene which is an important chemical for use in the production of polypropylene. These commercial processes are performed in the presence catalyst to produce the desired hydrocarbons to be used as raw materials for various chemical products.

### BRIEF SUMMARY

In accordance with an exemplary embodiment, a catalytic composite is disclosed. The catalytic composite comprises a first component, a second component, and a third component, all supported on an alumina support. The first component is selected from Group VIII noble metal components and combinations thereof. The second component is selected from one or more of alkali and alkaline earth metal components. The third component is selected from one or more of tin, germanium, lead, indium, gallium, and thallium. The alumina support of the catalytic composite comprises delta alumina. The catalytic composite comprising delta alumina is characterized by an X-ray diffraction pattern comprising at least three 2θ diffraction angle peaks between 32.0° and 70.0°. The at least three 2θ diffraction angle peaks comprise a first 2θ diffraction angle peak of 32.7°±0.4°, a second 2θ diffraction angle peak of 50.8°±0.4°, and a third 2θ diffraction angle peak of 66.7°±0.8°, wherein the second 2θ diffraction angle peak has an intensity of less than about 0.06 times the intensity of the third 2θ diffraction angle peak. The alumina support of the catalytic composite of the present disclosure has a surface area greater than about 114 m²/g.

These and other features, aspects, and advantages of the present disclosure will become better understood upon consideration of the following detailed description, drawings and appended claims.

### BRIEF DESCRIPTION OF THE DRAWINGS

The various embodiments are described in conjunction with the following figures wherein like numerals denote like elements.

FIG. 1 shows an X-ray diffraction pattern for the delta alumina support of the catalytic composite in accordance with the present disclosure.

FIG. 2 is a graph showing a comparative study of the activity and the stability of catalytic composite of the present disclosure with respect to a reference catalytic composite comprising a theta alumina support according to Example 1.

FIG. 3 shows an X-ray diffraction patterns of the delta alumina support of the catalytic composite of the of the present disclosure, a reference gamma alumina support, and a reference theta alumina support according to Example 2.

### DETAILED DESCRIPTION

A catalytic composite, a hydrocarbon conversion process using the catalytic composite, and a method of preparing the catalytic composite is disclosed. The alumina support of the catalytic composite is characterized by a surface area greater than about 114 m²/g and an improved average piece crush strength (PCS) compared to theta alumina support. The alumina support imparts multipronged benefits to the catalytic composite, for example a surface area of greater than about 114 m²/g of the alumina support leads to improved performance. Also, an improved average piece crush strength of the alumina support may help in reducing catalyst attrition and deterioration to fines. The alumina support of the present disclosure provides durability and ease of handling to the catalytic composite.

Embodiments of the present disclosure are described below, and such description is not intended to be limiting.

In accordance with an embodiment of the present disclosure, a catalytic composite is disclosed. The catalytic composite may comprise a first component selected from Group VIII noble metal components and combinations thereof, a second component selected from one or more of alkali and alkaline earth metal components, and a third component selected from one or more of tin, germanium, lead, indium, gallium, and thallium. The first component, the second component, and the third component are all supported on an alumina support comprising delta alumina.

The catalytic composite comprising delta alumina is characterized by a unique X-ray powder diffraction pattern. The unique X-ray powder diffraction pattern of the catalytic composite comprising delta alumina, having at least the d-spacings and relative intensities is set forth in Table A below:

TABLE A

| 2Θ | d (Å) | I/Io | I/I₀ % |
|---|---|---|---|
| 32.6°-32.8° | 2.7 | 39.8-64.8 | s |
| 44.8°-45.9° | 2.0 | 59.7-70.3 | s |
| 46.4°-47.6° | 1.9 | 34.8-52.1 | s |
| 50.7°-50.8° | 1.8 | 2.1-6.0 | w |
| 66.8°-67.4° | 1.4 | 100.0 | s |

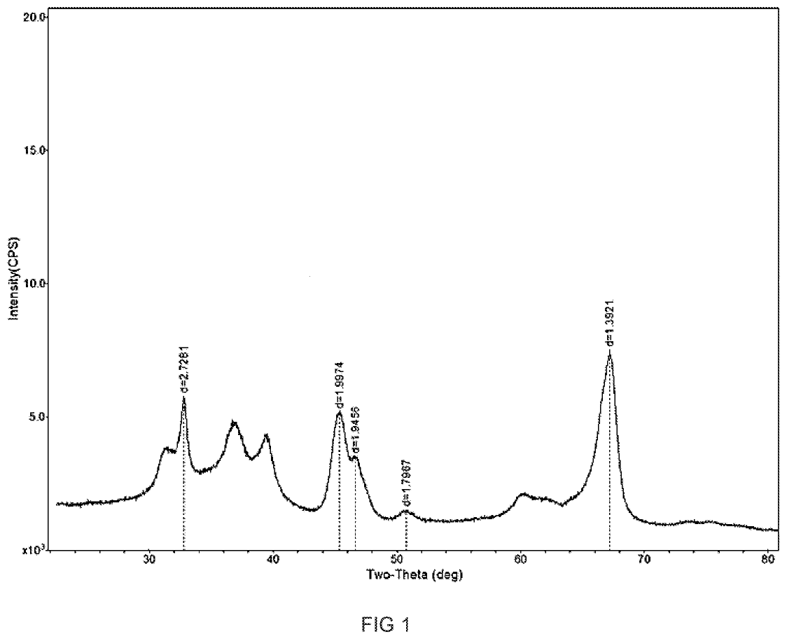

The X-ray powder diffraction pattern of the catalytic composite comprising delta alumina of the present disclosure is shown in FIG. 1. The unique X-ray powder diffraction pattern of the catalytic composite comprising delta alumina includes at least three 2θ diffraction angle peaks between 32.0° and 70.0°. The at least three 2θ diffraction angle peaks the of the catalytic composite comprise a first 2θ diffraction angle peak of 32.7°±0.4°, a second 2θ diffraction angle peak of 50.8°±0.4°, and a third 2θ diffraction angle peak of 66.7°±0.8°. The third 2θ diffraction angle peak of the X-ray powder diffraction pattern of the catalytic composite comprising delta alumina has the highest intensity as compared to the first 2θ diffraction angle peak and the second 2θ diffraction angle peak. Also, the first 2θ diffraction angle peak of the catalytic composite comprising delta alumina has an intensity of about 0.3 times to about 0.7 times the intensity of the third 2θ diffraction angle peak. It is also shown in FIG. 1 that the unique X-ray powder diffraction pattern of the catalytic composite comprising delta alumina has a weak peak at the second 2θ diffraction angle peak of 50.8°±0.4°. Also, the unique X-ray powder diffraction pattern of the catalytic composite comprising delta alumina has a visually apparent peak splitting between the diffraction angles (2θ) of about 43°±0.4° to about 49°±0.4° 20.

In an exemplary embodiment, the X-ray powder diffraction pattern of the catalytic composite comprising delta alumina has a weak peak at the second 2θ diffraction angle peak having an intensity of less than about 0.06 times the intensity of the third 2θ diffraction angle peak. The X-ray powder diffraction pattern of the catalytic composite comprising delta alumina also has a single peak in between the diffraction angles (2θ) of 50°±0.4° to 52°±0.4°.

Referring to the catalytic composite of the present disclosure, the first component is well dispersed throughout the catalytic composite. The catalytic composite may comprise the first component in an amount from about 0.01 weight percent to about 5.0 weight percent, or from about 0.1 weight percent to about 1.0 weight percent, or from about 0.2 weight percent to about 0.6 weight percent, calculated on an elemental basis of the final catalytic composite. In an exemplary embodiment, Group VIII noble metal may be selected from platinum, palladium, iridium, rhodium, osmium, ruthenium, or combinations thereof.

The first component, selected from the Group VIII noble metal components and combinations thereof, may be incorporated in the catalytic composite in any suitable manner such as, for example, by coprecipitation or cogellation, ion exchange or impregnation, or deposition from a vapor phase or from an atomic source or by like procedures either before, while, or after other catalytic components are incorporated. In an exemplary embodiment, the first component may be incorporated in the catalytic composite by impregnating the alumina support with a solution or a suspension of a decomposable compound of the first component. For example, platinum may be added to the support by commingling the latter with an aqueous solution of chloroplatinic acid. Another acid, for example, nitric acid or other optional components, may be added to the impregnating solution to further assist in evenly dispersing or fixing the first component in the catalytic composite.

The second component of the catalytic composite may be selected from one or more of alkali and alkaline earth metal components. In an exemplary embodiment, the second component of the catalytic composite may be selected from one or more of cesium, rubidium, potassium, sodium, and lithium. In another exemplary embodiment, the second component of the catalytic composite may be selected from one or more of barium, strontium, calcium, and magnesium. The second component may also be selected from either or both of these groups. In yet another exemplary embodiment, potassium, may be used as the second component. It is believed that the alkali and the alkaline earth component exists in the final catalytic composite in an oxidation state above that of the elemental metal. The alkali and alkaline earth component may be present as a compound such as oxide, for example, or combined with the support or with the other catalytic components.

The second component may also be well dispersed throughout the catalytic composite. The catalytic composite may comprise the second component in an in an amount from about 0.01 weight percent to about 5.0 weight percent, or from about 0.1 weight percent to about 2.0 weight percent, or from about 0.5 weight percent to about 1.5 weight percent, calculated on an elemental basis of the final catalytic composite.

The second component, selected from one or more of the alkali or alkaline earth metal components or mixtures thereof, may be incorporated in the catalytic composite in any suitable manner such as, for example, by coprecipitation or cogellation, by ion exchange or impregnation, or by like procedures either before, while, or after other catalytic components are incorporated. In an exemplary embodiment, the second component may be incorporated in the catalytic composite by impregnating the support with a solution of potassium hydroxide. In another exemplary embodiment, the second component may be incorporated in the catalytic composite by impregnating the support with a solution of potassium chloride.

The third component of the catalytic composite is a modifier metal component selected from tin, germanium, lead, indium, gallium, thallium, or mixtures thereof. The third component may be incorporated in the catalytic composite in any suitable manner. In an exemplary embodiment, the third component may be incorporated in the catalytic composite by impregnation.

The modifier metal component may be uniformly dispersed throughout the catalytic composite. This uniform dispersion can be achieved in a number of ways including impregnation of the catalyst with a modifier metal component containing solution, and incorporating the modifier metal component into the catalyst during catalyst support formulation. In the latter method, the modifier metal component may be added to the refractory oxide support during its preparation. In the case where the catalyst is formulated from a solution of the desired refractory oxide or precursor, the modifier metal may be incorporated into the solution before the catalyst was shaped. If the catalyst was formulated from a powder of the desired refractory oxide or precursor, the modifier may be added again prior to the shaping of the catalyst in the form of a dough into a particle. Incorporating the modifier metal into the catalyst support during its preparation may uniformly distribute the modifier metal throughout the catalyst.

The third component may be incorporated in the catalytic composite in any suitable manner such as by coprecipitation or cogellation with the carrier material, ion-exchange with the carrier material or impregnation of the carrier material at any stage in the preparation. In an embodiment where the third component is tin. The tin component may be incorporated into the catalytic composite by coprecipitating the tin component during the preparation of the carrier material. In this case, a suitable soluble tin compound such as stannous or stannic halide may be added to the alumina hydrosol, followed by combining the hydrosol with a suitable gelling agent and dropping the resulting mixture into an oil bath. After the calcination step, the resulting carrier material comprises an intimate combination of alumina and stannic oxide. In another embodiment, the tin component may be incorporated into the catalytic composite by using a soluble, decomposable compound of tin to impregnate the carrier material. Thus, a tin component may be added to the carrier material by commingling the latter with an aqueous solution of a suitable tin salt or water soluble compound of tin such as stannous bromide, stannous chloride, stannic chloride, stannic chloride pentahydrate, stannic chloride tetrahydrate, stannic chloride trihydrate, stannic chloride diamine, stannic trichloride bromide, stannic chromate, stannous fluoride, stannic fluoride, stannic iodide, stannic sulfate, stannic tartrate, and the like compounds. In an exemplary embodiment, a tin chloride compound, such as stannous or stannic chloride may be used. In general, the tin component can be impregnated either prior to, simultaneously with, or after the platinum group and/or germanium components are added to the carrier material.

The catalytic composite may comprise the third component in an amount from about 0.01 weight percent to about 5.0 weight percent, or from about 0.05 weight percent to about 0.5 weight percent, or from about 0.1 weight percent to about 0.3 weight percent, calculated on an elemental basis of the final catalytic composite.

The third component may exist within the catalytic composite as a compound such as oxide, sulfide, halide, oxychloride, aluminate, etc., or in combination with the support or other ingredients/components of the catalytic composite. In an exemplary embodiment, the third component of the catalytic composite may be tin. Some or all of the tin component may be present in the catalytic composite in an oxidation state above that of the elemental metal. The tin component may be used in an amount sufficient to result in the final catalytic composite containing, on an elemental basis, about 0.01 to about 5.0 weight percent tin, or from about 0.05 weight percent to about 0.5 weight percent tin, or from about 0.1 weight percent to about 0.3 weight percent tin.

Suitable tin salts or water-soluble compounds of tin which may be used include stannous bromide, stannous chloride, stannic chloride, stannic chloride pentahydrate, stannic chloride tetrahydrate, stannic chloride trihydrate, stannic chloride diamine, stannic trichloride bromide, stannic chromate, stannous fluoride, stannic fluoride, stannic iodide, stannic sulfate, stannic tartrate, and the like compounds. In an exemplary embodiment, a tin chloride compound, such as stannous or stannic chloride may be used. The third component of the catalyst may be composited with the support in any sequence. Thus, the first or the second component may be impregnated on the support followed by sequential surface or uniform impregnation of the third component. Alternatively, the third component may be surface impregnated or uniformly impregnated on the support followed by impregnation of the other catalytic component.

The catalytic composite may also comprise a halogen component. The halogen component may be fluorine, chlorine, bromine, or iodine, or mixtures thereof. In an exemplary embodiment, chlorine may be used as the halogen component. The halogen component may be present in a combined state with the porous support and the alkali component. The halogen component may also be well dispersed throughout the catalytic composite. The halogen component may be present in an amount from more than 0.01 weight percent to about 6 weight percent, calculated on an elemental basis, of the final catalytic composite.

The halogen component may be incorporated in the catalytic composite in any suitable manner, either during the preparation of the support or before, while, or after other catalytic components are incorporated. For example, the alumina solution that may be utilized to form the aluminum support may contain halogen and thus contribute at least some portion of the halogen content in the final catalytic composite. Also, the halogen component or a portion thereof may be added to the catalytic composite during the incorporation of the support with other catalyst components, for example, by using chloroplatinic acid to impregnate the platinum component. The halogen component or a portion thereof may be added to the catalytic composite by contacting the catalyst with the halogen or a compound or a solution containing the halogen before or after other catalyst components are incorporated with the support. The halogen component or a portion thereof may be added during the heat treatment of the catalytic composite. Suitable compounds containing the halogen include acids containing the halogen, for example, hydrochloric acid. Or, the halogen component or a portion thereof may be incorporated by contacting the catalytic composite with a compound or a solution containing the halogen in a subsequent catalyst regeneration step. In the regeneration step, carbon deposited on the catalyst as coke during use of the catalyst in a hydrocarbon conversion process is burned off and the catalyst and the platinum group component on the catalyst is redistributed to provide a regenerated catalyst with performance characteristics much like the fresh catalyst. The halogen component may be added during the carbon burn step or during the Group VIII noble metal component redispersion step, for example, by contacting the catalyst with a chlorine gas. Also, the halogen component may be added to the catalytic composite by adding the halogen or a compound or solution containing the halogen, such as propylene dichloride, for example, to the hydrocarbon feed stream or to the recycle gas during operation of the hydrocarbon conversion process. The halogen may also be added as chlorine gas (Cl₂).

The support of catalytic composite is an alumina support comprising delta alumina. The alumina support of the catalytic composite has a surface area greater than about 114 m²/g. The alumina support may comprise delta alumina in an amount greater than about 75 weight percent. The alumina support may be prepared by any suitable manner from synthetic or naturally occurring raw materials. Also, the alumina support may be formed in any desired shape such as spheres, pills, cakes, extrudates, powders, granules, and other shapes, and it may be utilized in any particle size. In an exemplary embodiment, the shape of alumina support is spherical. A particle size of about ⅛ inch (3 mm) in diameter or about 1/16 inch (1.6 mm) in diameter may be used. A larger particle size may also be utilized.

The spherical alumina support may be prepared by converting an alumina metal into an alumina solution by reacting it with a suitable peptizing agent and water. Then, a mixture of the alumina solution may be dropped into an oil bath to form spherical particles of the alumina gel. Other shapes of the alumina support may also be prepared by conventional methods. After the alumina optionally containing the co-formed third component is shaped, it may be dried and calcined.

In accordance with the present disclosure, calcination of the alumina base at a closely controlled temperature may be directed towards imparting the alumina support with the desired characteristics or properties. The surface area of the alumina support is greater than about 114 m²/g or greater than about 115 m²/g or greater than about 120 m²/g. Also, the average piece crush strength of the alumina support is greater than the usual/conventional theta alumina support. These characteristics may be imparted into the alumina support of the present disclosure by a final calcination of an alumina precursor at a temperature ranging from about 800° C. (1472° F.) to about 1000° C. (1832° F.) or about 800° C. (1472° F.) to about 950° C. (1742° F.). The final calcination step should be operated at conditions sufficient to convert the alumina precursor into delta alumina which imparts the desired characteristics to the alumina support of the instant catalytic composite. Such conditions would include a calcination temperature closely controlled between from about 800° C. (1472° F.) to about 950° C. (1742° F.).

The surface area of the alumina support may be measured by nitrogen adsorption as per BET surface area measurement method. For nitrogen adsorption BET measuring device ASAP 2010 from Micromeritics is used and multi-point BET measurement technique of DIN 66131 is used. A sample amount in the range of 0.1 g to 1.0 g may be used. For surface area measurement, 5 measurement points or more can be taken within a relative pressure range (P/PO) of from 0.05 to 0.25 of the adsorption isotherm. In an embodiment, the alumina support has a surface area greater than about 114 m²/g or greater than about 115 m²/g or greater than about 120 m²/g. In an exemplary embodiment, the alumina support has a surface area from about 114 m²/g to about 150 m²/g.

The alumina support may comprise essentially delta alumina. By “essentially delta alumina”, it is meant that the alumina support comprises delta alumina in an amount greater than about 99 weight percent, or greater than about 97 weight percent, or greater than about 95 weight percent. The alumina crystallites of the alumina support may comprise 100% delta alumina crystallites. Any remaining crystallites of alumina may be present in the form of theta alumina or gamma alumina. However, other forms of alumina crystallites known in the art may also be present. In an embodiment, the alumina support may comprise theta alumina in an amount no greater than about 1 weight percent, or no greater than about 3 weight percent, or no greater than about 5 weight percent. The alumina support should include no greater than about 5 weight percent of theta alumina.

The delta alumina form of crystalline alumina may be produced from the alumina precursor by closely controlling the maximum calcination temperature experienced by the catalyst support. Any suitable alumina precursor may be used for producing the alumina support of the present disclosure. In one embodiment, the alumina precursor may be gamma alumina. In another exemplary embodiment, the alumina precursor may be boehmite. Instead of typical theta alumina conversion at a temperature of 1050° C. (1922° F.), the alumina support of the present disclosure, comprising delta alumina, is obtained by calcining the alumina precursor at a tightly controlled calcination temperature from about 800° C. (1472° F.) to about 1000° C. (1832° F.). The calcination temperature of the delta alumina support of the present disclosure is well below the calcination temperature of 1050° C. (1922° F.) for obtaining theta alumina. Applicants have found that to produce the delta alumina support with the desired characteristics such as durability and ease of handling, the calcination temperature should be tightly controlled to be from about 800° C. (1472° F.) to about 1000° C. (1832° F.) or about 800° C. (1472° F.) to about 950° C. (1742° F.) or about 900° C. (1652° F.) to about 950° C. (1742° F.) or about 900° C. (1652° F.) to about 940° C. (1724° F.). Such calcination temperatures produce alumina support comprising delta alumina crystallites. Also, such calcination temperatures provide a delta alumina support having a surface area greater than about 114 m²/g, or greater than about 115 m²/g, or greater than about 120 m²/g. The average piece crush strength of the alumina support is also better than the usual/conventional theta alumina support. A delta alumina support prepared in this way and having the surface area greater than about 114 m²/g or greater than about 115 m²/g, or greater than about 120 m²/g meets the desired durability and ease of handling. In an exemplary embodiment, an alumina precursor may be calcined for a time from about 10 minutes to about 180 minutes at a temperature from about 900° C. (1652° F.) to about 950° C. (1742° F.) to produce the alumina support comprising delta alumina.

Generally, average piece crush strength plays an important role for durability and handling of the catalytic composite. Under given operating conditions in a reactor, higher piece crush strength leads to less catalyst attrition and deterioration to fines. Catalysts with poor piece crush strength have propensity for more often fracturing and generating dust and catalyst fines that can become trapped against, for example, reactor screens. The dust and fines can lead to blocked flow of reactants and products, which often may require the unit to shut down for screen cleaning. For a given operating conditions, frequent catalyst make-up volumes, in order to replace catalyst inventory lost to fines, dust, or cracked chips, may be required, which is costly both in material costs and operational costs. The average piece crush strength of the delta alumina support can be measured by ASTM D4179 or an equivalent method. The delta alumina support of the present disclosure prepared under calcination temperatures from about 900° C. (1652° F.) to about 950° C. (1742° F.) reported an improved average piece crush strength compared to the theta alumina support. An improved average piece crush strength may lead to catalytic composites which generate lesser dust and catalyst fines and do not fracture easily under given operating conditions.

After all the components have been composited or combined with the alumina support comprising delta alumina, the resulting catalytic composite will generally be dried at a temperature of from about 90° C. (194° F.) to about 320° C. (608° F.) for a period of typically about 1 hour to 24 hours or more. The dried catalytic composite may be further calcined at a temperature of about 320° C. (608° F.) to about 600° C. (1112° F.) for a period of typically about 0.5 hours to about 10 hours or more. Typically, chlorine-containing compounds are added to air to prevent sintering of catalyst metal components. This final calcination typically does not affect the alumina crystallites or particularly the desired properties of the surface area and the average piece crush strength of the alumina support or the catalytic composite. Thereafter, the calcined catalytic composite is typically subjected to a reduction step before use in the hydrocarbon conversion process. This reduction step may be performed at a temperature of about 230° C. (446° F.) to about 650° C. (1202° F.) for a period of about 0.5 hours to about 10 hours or more in a reducing environment, e.g. dry hydrogen, the temperature and time being selected to be sufficient to reduce substantially all of the noble metal group component to the elemental metallic state.

The catalytic composite of the present disclosure may be used as a hydrocarbon conversion catalyst in a hydrocarbon conversion process. The hydrocarbon which is to be converted is contacted with the catalytic composite at hydrocarbon conversion conditions. The catalytic composite may be used in various hydrocarbon conversion processes including but not limited to dehydrogenation, oxidative dehydrogenation, hydrogenation, transfer hydrogenation, aromatization, and reforming processes. Operating conditions for the dehydrogenation processes may comprise a temperature of from about 200° C. (392° F.) to 1000° C. (1832° F.), a pressure of from 25 kPa absolute (3.6 psia) to about 2550 kPa absolute (370 psia), and a liquid hourly space velocities of from about 0.1 hr⁻¹ to about 200 hr⁻¹. The reforming process may be operated at a temperature of from about 400° C. (752° F.) to about 560° C. (1040° F.), a pressure of from about 100 kPa (14 psia) to 6000 kPa (870 psia), and a liquid hourly space velocity of from about 0.2 hr⁻¹ to about 20 hr⁻¹.

In an exemplary embodiment, the hydrocarbon conversion process is dehydrogenation process. In the dehydrogenation process, a feed comprising dehydrogenatable hydrocarbons may be contacted with the catalytic composite of the present disclosure in a dehydrogenation zone maintained at dehydrogenation conditions. The feed may be contacted with the catalytic composite in a fixed catalyst bed system, a moving catalyst bed system, a fluidized bed system, or in a batch-type operation. A fixed bed system is typically used in the dehydrogenation process. In the fixed bed system, a hydrocarbon feed stream is preheated to the desired reaction temperature and then passed into the dehydrogenation zone containing a fixed bed of the catalytic composite. The dehydrogenation zone may itself comprise one or more separate reaction zones with heating means therebetween to ensure that the desired reaction temperature can be maintained at the entrance to each reaction zone. The feed may be contacted with the catalytic composite bed in either upward, downward, or radial flow fashion. Usually, radial flow is opted for commercial scale reactors. The feed may be in a liquid phase, a mixed vapor-liquid phase, or a vapor phase when the feed contacts the catalytic composite. Typically, the feed is maintained in the vapor phase.

The feed that may be used in the dehydrogenation process include dehydrogenatable hydrocarbons having from 2 to 30 or more carbon atoms including paraffins, alkylaromatics, naphthenes, and olefins. One group of hydrocarbons which can be dehydrogenated with the catalytic composite includes the group of normal paraffins having from 2 to 30 or more carbon atoms. The catalytic composite may be used for dehydrogenating paraffins having from 2 to 15 or more carbon atoms to the corresponding monoolefins or for dehydrogenating monoolefins having from 3 to 15 or more carbon atoms to the corresponding diolefins. The catalytic composite is especially useful in the dehydrogenation of C₂-C₆ paraffins, primarily propane and butanes, to monoolefins.

Generally, for normal paraffins, the lower the molecular weight, the higher the temperature required for comparable conversion. The pressure in the dehydrogenation zone is maintained as low as practicable, consistent with equipment limitations, to maximize the chemical equilibrium advantages. In an exemplary embodiment, dehydrogenation conditions may include a temperature of from about 400° C. (752° F.) to about 900° C. (1652° F.), a pressure of from about 1 kPa absolute (0.14 psia) to 1014 kPa absolute (147 psia), and a liquid hourly space velocity (LHSV) of from about 0.1 hr⁻¹ to 100 hr⁻¹.

An effluent stream from the dehydrogenation zone generally will contain unconverted dehydrogenatable hydrocarbons, hydrogen, and the products of dehydrogenation reactions. The effluent stream is typically cooled and passed to a hydrogen separation zone to separate a hydrogen-rich vapor phase from a hydrocarbon-rich liquid phase. Generally, the hydrocarbon-rich liquid phase is further separated by means of either a suitable selective adsorbent, a selective solvent, a selective reaction or reactions, or by means of a suitable fractionation scheme. Unconverted dehydrogenatable hydrocarbons are recovered and may be recycled to the dehydrogenation zone. Products of the dehydrogenation reactions are recovered as final products or as intermediate products in the preparation of other compounds.

The dehydrogenatable hydrocarbons may be admixed with a diluent material before, while, or after being passed to the dehydrogenation zone. The diluent material may be hydrogen, steam, methane, ethane, carbon dioxide, nitrogen, argon, and the like or a mixture thereof. Typically, hydrogen and steam are used as diluents. Ordinarily, when hydrogen or steam is utilized as the diluent, it is utilized in amounts sufficient to ensure a diluent-to-hydrocarbon mole ratio of about 0.1:1 to about 40:1. The diluent stream passed to the dehydrogenation zone will typically comprise a recycled diluent separated from the effluent stream of the dehydrogenation zone in a separation zone.

A combination of diluents, such as steam with hydrogen, may also be employed. When hydrogen is the primary diluent, water or a material which decomposes at dehydrogenation conditions to form water such as but not limited to an alcohol, or an ether, may be added to the dehydrogenation zone, either continuously or intermittently, in an amount to provide, calculated on the basis of equivalent water, about 1 to about 20,000 weight ppm of the hydrocarbon feed stream. About 1 to about 10,000 weight ppm of water addition may be used when dehydrogenating paraffins having from 6 to 30 or more carbon atoms.

To be commercially successful, a dehydrogenation catalyst or catalytic composite should exhibit high activity, high selectivity, and good stability. Activity is a measure of the catalyst's ability to convert reactants into products at a specific set of reaction conditions, that is, at a specified temperature, pressure, contact time, and concentration of diluent such as hydrogen, if any. For dehydrogenation catalyst activity, the conversion or disappearance of paraffins in percent relative to the amount of paraffins in the feedstock is measured. Selectivity is a measure of the catalyst's ability to convert reactants into the desired product or products relative to the amount of reactants converted. For catalyst selectivity, the amount of olefins in the product, in mole percent, relative to the total moles of the paraffins converted is measured. Stability is a measure of the rate of change with time on stream of the activity and selectivity parameters the smaller rates implying the more stable catalysts. The catalytic composite of the present disclosure comprises a delta alumina support having a surface area greater than about 114 m²/g. The catalytic composite with delta alumina support of the present disclosure has improved performance including but not limited to, reduced catalyst attrition and deterioration to fines, durability and ease of handling under given operating conditions. These advantages including activity and stability of the catalytic composite of the present disclosure are demonstrated in examples.

The structure or the presence of delta alumina for the alumina support of the catalytic composite of the present disclosure was determined by X-ray analysis. The X-ray patterns listed herein above and in the examples, were obtained using standard X-ray powder diffraction techniques. The radiation source was a high-intensity X-ray tube operated at 45 kV and 35 mA. The diffraction pattern from the copper K-alpha radiation was obtained by appropriate computer based techniques. Flat compressed powder samples were continuously scanned at 2° to 80° (2θ). Interplanar spacings (d) in Angstrom units were obtained from the position of the diffraction peaks expressed as 0, where 0 is the Bragg angle as observed from digitized data. Intensities were determined from the integrated area of diffraction peaks after subtracting background, “Io” being the intensity of the strongest line or peak, and “I” being the intensity of each of the other peaks.

As will be understood by those skilled in the art the determination of the diffraction angles (2θ) is subject to both human and mechanical error, which in combination can impose an uncertainty of about ±0.4° on each reported value of 2θ. This uncertainty is, of course, also manifested in the reported values of the d-spacings, which are calculated from the 2θ values. This imprecision is general throughout the art and is not sufficient to preclude the differentiation of the present crystalline materials from each other and from the compositions of the prior art. In some of the X-ray patterns reported, the relative intensities of the d-spacings are indicated by the notations vs, s, m, w, and vw which represent very strong, strong, medium, weak and very weak, respectively. In terms of 100×I/Io, the above designations are defined as:
vw<1, w=1-10; m=10-32; s=32-100; and vs >100

In certain instances the purity of a synthesized product may be assessed with reference to its X-ray powder diffraction pattern. Thus, for example, if a sample is stated to be pure, it is intended only that the X-ray pattern of the sample is free of lines attributable to crystalline impurities, not that there are no amorphous materials present.

The following examples are introduced to further describe the catalytic composite and the process of the present disclosure. These examples are intended as an illustrative embodiment and should not be considered to restrict the otherwise broad interpretation of the disclosure as set forth in the claims appended hereto.

### Example 1

The efficacy of the catalytic composite in a dehydrogenation process was demonstrated. Firstly, a spherical alumina support was prepared by oil-drop method. An alumina hydroxyl chloride solution was formed by dissolving substantially pure aluminum pellets in a hydrochloric acid solution. Then, hexamethylenetetramine was added to the solution followed by gelling the resulting solution by dropping it into an oil bath to form spherical particles of an alumina hydrogel. For adding a tin component, a tin component precursor was commingled with the alumina hydrosol followed by gelling the hydrosol. The tin component in this case was uniformly distributed throughout the catalyst particles. The resulting particles were aged and washed with an ammoniacal solution and finally dried, calcined, and steamed to form spherical particles of delta alumina. For this, the catalyst particles were dried at a temperature of about 93° C. (200° F.) to about 316° C. (601° F.) for about 2 hours and calcined at a temperature of about 800° C. (1472° F.) to about 950° C. (1742° F.). The calcined tin-containing catalyst particles were then contacted with a chloroplatinic acid solution and a potassium chloride solution to uniformly impregnate the alumina base with platinum and potassium. After impregnation, the catalytic composite was heat-treated in air at a temperature of about 500° C. (932° F.) for 4 hours in the presence of 3% steam and chlorine-containing gases, followed by reduction in hydrogen at about 550° C. (1022° F.) for about 2 hours. The surface area of the alumina support was measured by nitrogen adsorption method. Three catalytic composites, A, B, and C were prepared in accordance with the aforesaid method comprising 0.2 to 0.6 weight percent platinum, 0.1 to 0.3 weight percent tin, and 0.5 to 1.5 weight percent potassium. The surface area of the alumina support of the catalytic composites A, B, and C was measured by nitrogen adsorption method. The surface areas of the alumina support for the catalytic composites A, B, and C were found to be about 114 m²/g, about 120 m²/g and about 130 m²/g respectively.

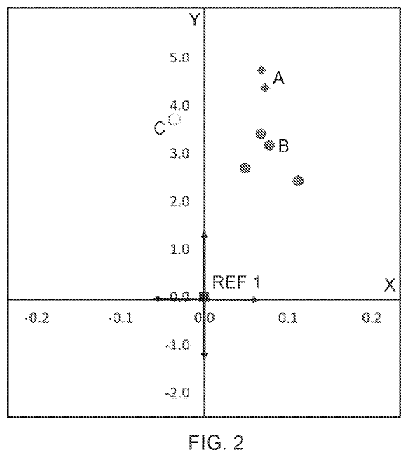

The catalytic composites A, B, and C were tested in a dehydrogenation process to dehydrogenate propane to produce propylene. The operating conditions of the dehydrogenation process included a liquid hourly space velocity (LHSV) of 30 hr⁻¹, a pressure of 135 kPa (5 psig) and a feed temperature of 655° C. (1210° F.). A gradual increase in temperature was used to attain the feed temperature of 655° C. (1210° F.). The hydrocarbon feed was fed over each of the catalytic composites for 18 hours. The maximum conversion of the feed was achieved in 3 to 4 hour on stream (HOS). The same test was performed over a reference catalyst bed containing theta alumina support having a surface area of 90 m²/g. The maximum conversion of the feed achieved with each of the catalytic composites A, B, and C of the present disclosure was compared with the maximum conversion of the feed achieved with the reference catalyst containing theta alumina support. The difference between the maximum conversion of the feed achieved with the catalytic composite having delta alumina support and the maximum conversion of the feed achieved with the catalytic composite having theta alumina support is the delta activity (error±1.3) which is plotted on Y-axis in FIG. 2. The delta activity was calculated for the catalytic composites A, B, and C. Delta stability of the catalytic composite was also calculated. The stability of the catalytic composite was calculated as below:

$$Stability = \frac{Conversion of the feed at 5 H O S - Conversion of the feed at 15 H O S}{10 hour}$$

The stability of catalytic composites A, B, and C were calculated using the above formula. The stability of the reference catalyst containing theta alumina support was also calculated using the above formula. The difference between the stability of the catalytic composite comprising delta alumina and the stability of the reference catalyst containing theta alumina is delta stability. The delta stability (error±0.6) of catalytic composites A, B, and C are plotted on the X-axis in FIG. 0.2. In FIG. 2, the reference catalyst containing theta alumina support is shown as “REF 1” which is the reference point (0, 0). It is evident from FIG. 2 that the catalytic composites A, B, and C of the present disclosure showed a positive delta activity compared to the reference catalyst containing theta alumina support. The delta stability of the catalytic composites A, B, and C of the present disclosure was also found better and within the error bar of ±0.6 compared to the reference catalyst as shown in FIG. 2.

### Example 2

### X-Ray Determination:

To determine the X-ray pattern, three new catalytic composites D, E, and F comprising delta alumina were prepared using the method of example of 1. As measured by nitrogen adsorption method, the surface areas of the alumina support for the catalytic composites D, E, and F were found to be about 115 m²/g, about 140 m²/g, and about 150 m²/g respectively. X-ray analysis of the three new catalytic composites D, E, and F and the catalytic composite B of example 1 was performed. For comparison, an X-ray analysis for the reference catalyst containing theta alumina support “REF 1” of example 1 was also performed to collect the X-ray pattern of the reference catalyst. Another X-ray analysis for another reference catalyst comprising gamma alumina support “REF 2” was also performed for comparison. The results of the X-ray analysis of all the catalysts are listed herein TABLE B below:

TABLE B

| Catalyst | 2Θ | d (Å) | I/Io | I/Io |
|---|---|---|---|---|
| B | 32.8° | 2.7 | 64.3 | s |
| | 45.3° | 2.0 | 62.0 | s |
| | 46.6° | 1.9 | 37.1 | s |
| | 50.8° | 1.8 | 5.6 | w |
| | 67.2° | 1.4 | 100.0 | s |
| D | 32.8° | 2.7 | 64.8 | s |
| | 45.4° | 2.0 | 59.7 | s |
| | 46.6° | 1.9 | 34.8 | s |
| | 50.8° | 1.8 | 6.0 | w |
| | 67.2° | 1.4 | 100.0 | s |
| E | 32.7° | 2.7 | 50.7 | s |
| | 45.4° | 2.0 | 68.5 | s |
| | 46.4° | 2.0 | 46.4 | s |
| | 50.8° | 1.8 | 4.6 | w |
| | 67.1° | 1.4 | 100.0 | s |
| F | 32.7° | 2.7 | 39.8 | s |
| | 45.5° | 2.0 | 70.3 | s |
| | 46.6° | 1.9 | 52.1 | s |
| | 50.8° | 1.8 | 2.1 | w |
| | 67.0° | 1.4 | 100.0 | s |
| REF 1 | 32.8° | 2.7 | 133.0 | vs |
| (theta | 44.8° | 2.0 | 65.0 | s |
| alumina) | 47.6° | 1.9 | 40.8 | s |
| | 50.7° | 1.8 | 15.8 | m |
| | 67.4 | 1.4 | 100.0 | s |
| REF 2 | 32.6 | 2.7 | 31.0 | m |
| (gamma | 45.9 | 2.0 | 84.2 | s |
| alumina) | 66.8 | 1.4 | 100.0 | s |

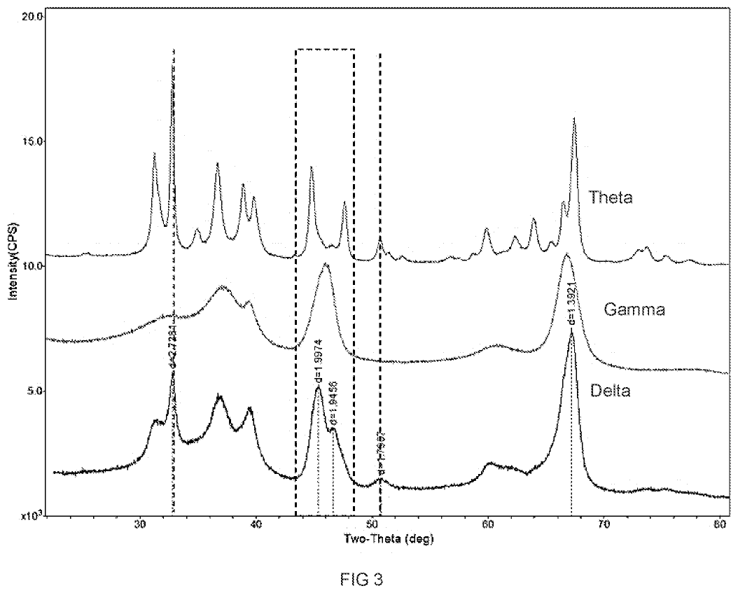

The X-ray powder diffraction pattern for the catalytic composites B, D, E, and F comprising delta alumina is combinedly shown in FIG. 3 as “Delta”. The X-ray powder diffraction patterns of the reference catalysts REF 1 and REF 2 comprising theta and gamma alumina support respectively are also shown in FIG. 3 as “Theta” and “Gamma” respectively. As shown, the X-ray powder diffraction pattern of the delta alumina support for the catalytic composites B, D, E, and F showed three distinct diffraction angle peaks, a first 2θ diffraction angle peak at 32.7°±0.4°, a second 2θ diffraction angle peak at 50.8°±0.4°, and a third 2θ diffraction angle peak at 66.7°±0.8°. Also, the second 2θ diffraction angle peak at 50.8°±0.4° had an intensity of less than about 0.06 times the intensity of the third 2θ diffraction angle peak at 66.7°±0.8° which showed the highest intensity compared to the first 2θ diffraction angle peak and the second 2θ diffraction angle peak. The second 2θ diffraction angle peak at 50.8°±0.4° was the weakest compared to the other two. The intensity of first 2θ diffraction angle peak at 32.7°±0.4° was found to be in between 0.3 times to about 0.7 times the intensity of the third 2θ diffraction angle peak at 66.7°±0.8°. Also, the X-ray powder diffraction pattern for the catalytic composites, B, D, E, and F showed visually apparent splitting of the broad peak(s) between the diffraction angles (2θ) of 43°±0.4° to 49°±0.4°.

As compared to the X-ray powder diffraction pattern for the catalytic composites comprising delta alumina, the X-ray powder diffraction pattern of the gamma alumina showed no 2θ diffraction angle peak at 50.8°±0.4°. Also, no visually apparent splitting of the broad peak(s) between the diffraction angles (2θ) of 43°±0.4° to 49°±0.4° was observed in the X-ray powder diffraction pattern of the gamma alumina. Contrary to the X-ray powder diffraction pattern for the delta alumina, the X-ray powder diffraction pattern of the theta alumina showed a highest 2θ diffraction angle peak at 32.7°±0.4°. Also, the X-ray powder diffraction pattern of the theta alumina had multiple 2θ diffraction angle peak in between 50°±0.4° to 52°±0.4°. No visually apparent splitting of the broad peak(s) between the diffraction angles (2θ) of 43°±0.4° to 49°±0.4° was observed in the X-ray powder diffraction pattern of the theta alumina. There were two separate/distinct peaks observed between the diffraction angles (2θ) of 43°±0.4° to 49°±0.4° in the X-ray powder diffraction pattern of the theta alumina as shown in FIG. 3. This observation was contrary to the peak splitting observed between the diffraction angles (2θ) of 43°±0.4° to 49°±0.4° in the X-ray powder diffraction pattern for the catalytic composites comprising delta alumina as shown in FIG. 3.

### SPECIFIC EMBODIMENTS

While the following is described in conjunction with specific embodiments, it will be understood that this description is intended to illustrate and not limit the scope of the preceding description and the appended claims.

A first embodiment of the present disclosure is a catalytic composite comprising a first component selected from Group VIII noble metal components and combinations thereof, a second component selected from one or more of an alkali and alkaline earth metal components, and a third component selected from one or more of tin, germanium, lead, indium, gallium, and thallium, all supported on an alumina support comprising delta alumina, the alumina support having an X-ray diffraction pattern comprising at least three 2θ diffraction angle peaks between 32.0° and 70.0°, wherein a first 2θ diffraction angle peak is at 32.7°±0.4°, a second 2θ diffraction angle peak is at 50.8°±0.4°, and a third 2θ diffraction angle peak is at 66.7°±0.8°, and wherein the second 2θ diffraction angle peak has an intensity of less than about 0.06 times the intensity of the third 2θ diffraction angle peak. An embodiment of the present disclosure is one, any or all of prior embodiments in this paragraph up through the first embodiment in this paragraph, wherein the third 2θ diffraction angle peak has the highest intensity compared to the first 2θ diffraction angle peak and the second 2θ diffraction angle peak. An embodiment of the present disclosure is one, any or all of prior embodiments in this paragraph up through the first embodiment in this paragraph, wherein the first 2θ diffraction angle peak has an intensity of about 0.3 times to about 0.7 times the intensity of the third 2θ diffraction angle peak. An embodiment of the present disclosure is one, any or all of prior embodiments in this paragraph up through the first embodiment in this paragraph, wherein the X-ray diffraction pattern has a single peak between the diffraction angles (2θ) of 50°±0.4° to 52°±0.4°. An embodiment of the present disclosure is one, any or all of prior embodiments in this paragraph up through the first embodiment in this paragraph, wherein the X-ray diffraction pattern has a peak splitting between the diffraction angles (2θ) of about 43°±0.4° to about 49°±0.4°. An embodiment of the present disclosure is one, any or all of prior embodiments in this paragraph up through the first embodiment in this paragraph, wherein the alumina support has a surface area greater than about 114 m²/g. An embodiment of the present disclosure is one, any or all of prior embodiments in this paragraph up through the first embodiment in this paragraph further comprising from about 0.01 weight percent to about 5.0 weight percent the first component, from about 0.01 weight percent to about 5.0 weight percent the second component, and from about 0.01 weight percent to about 5.0 weight percent the third component. An embodiment of the present disclosure is one, any or all of prior embodiments in this paragraph up through the first embodiment in this paragraph, wherein the first component is platinum. An embodiment of the present disclosure is one, any or all of prior embodiments in this paragraph up through the first embodiment in this paragraph, wherein the second component is potassium. An embodiment of the present disclosure is one, any or all of prior embodiments in this paragraph up through the first embodiment in this paragraph, wherein the third component is tin.

A second embodiment of the present disclosure is a hydrocarbon conversion process comprising contacting a feed at hydrocarbon conversion conditions with a catalytic composite to generate at least one product wherein the catalytic composite comprises a first component selected from Group VIII noble metal components and mixtures thereof, a second component selected from one or more of alkali and alkaline earth metal components, and a third component selected from one or more of tin, germanium, lead, indium, gallium, and thallium, supported on an alumina support comprising delta alumina having an X-ray diffraction pattern comprising at least three 2θ diffraction angle peaks between 32.0° and 70.0°, the at least three 2θ diffraction angle peaks comprise a first 2θ diffraction angle peak of 32.7°±0.4°, a second 2θ diffraction angle peak of 50.8°±0.4°, and a third 2θ diffraction angle peak of 66.7°±0.8°, wherein the second 2θ diffraction angle peak has an intensity of less than about 0.06 times the intensity of the third 2θ diffraction angle peak. An embodiment of the present disclosure is one, any or all of prior embodiments in this paragraph up through the second embodiment in this paragraph, wherein the third 2θ diffraction angle peak has the highest intensity compared to the first 2θ diffraction angle peak and the second 2θ diffraction angle peak. An embodiment of the present disclosure is one, any or all of prior embodiments in this paragraph up through the second embodiment in this paragraph, wherein the first 2θ diffraction angle peak has an intensity of about 0.3 times to about 0.7 times the intensity of the third 2θ diffraction angle peak. An embodiment of the present disclosure is one, any or all of prior embodiments in this paragraph up through the second embodiment in this paragraph, wherein the X-ray diffraction pattern of the alumina support comprising delta alumina has a single peak in between the diffraction angles (2θ) of 50°±0.4° to 52°±0.4°. An embodiment of the present disclosure is one, any or all of prior embodiments in this paragraph up through the second embodiment in this paragraph, wherein the X-ray diffraction pattern has a peak splitting between the diffraction angles (2θ) of about 43°±0.4° to about 49°±0.4°. An embodiment of the present disclosure is one, any or all of prior embodiments in this paragraph up through the second embodiment in this paragraph, wherein the alumina support has a surface area greater than about 114 m²/g. An embodiment of the present disclosure is one, any or all of prior embodiments in this paragraph up through the second embodiment in this paragraph, wherein the hydrocarbon conversion process is one or more of oxidative dehydrogenation, hydrogenation, transfer hydrogenation, aromatization, and reforming processes. An embodiment of the present disclosure is one, any or all of prior embodiments in this paragraph up through the second embodiment in this paragraph, wherein the hydrocarbon conversion process is a dehydrogenation process. An embodiment of the present disclosure is one, any or all of prior embodiments in this paragraph up through the second embodiment in this paragraph, wherein the catalytic composite comprises from about 0.01 weight percent to about 5.0 weight percent the first component, from about 0.01 weight percent to about 5.0 weight percent the second component, and from about 0.01 weight percent to about 5.0 weight percent the third component. An embodiment of the present disclosure is one, any or all of prior embodiments in this paragraph up through the second embodiment in this paragraph, wherein the first component is platinum, the second component is potassium, and the third component is tin.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A hydrocarbon conversion process comprising contacting a feed at hydrocarbon conversion conditions with a catalytic composite to generate at least one product wherein the catalytic composite comprises a first component selected from Group VIII noble metal components and mixtures thereof, a second component selected from one or more of alkali and alkaline earth metal components, and a third component selected from one or more of tin, germanium, lead, indium, gallium, and thallium, supported on an alumina support comprising delta alumina having an X-ray diffraction pattern comprising at least three 2θ diffraction angle peaks between 32.0° and 70.0°, the at least three 2θ diffraction angle peaks comprise a first 2θ diffraction angle peak of 32.7°±0.4°, a second 2θ diffraction angle peak of 50.8°±0.4°, and a third 2θ diffraction angle peak of 66.7°±0.8°, wherein the second 2θ diffraction angle peak has an intensity of less than about 0.06 times the intensity of the third 2θ diffraction angle peak.

2. The process of claim 1, wherein the third 2θ diffraction angle peak has the highest intensity compared to the first 2θ diffraction angle peak and the second 2θ diffraction angle peak.

3. The process of claim 1, wherein the first 2θ diffraction angle peak has an intensity of about 0.3 times to about 0.7 times the intensity of the third 2θ diffraction angle peak.

4. The process of claim 1, wherein the X-ray diffraction pattern of the alumina support comprising delta alumina has a single peak in between the diffraction angles (2θ) of 50°±0.4° to 52°±0.4°.

5. The catalytic composite of claim 1, wherein the X-ray diffraction pattern has a peak splitting between the diffraction angles (2θ) of about 43°±0.4° to about 49°±0.4°.

6. The process of claim 1, wherein the alumina support has a surface area greater than about 114 m²/g.

7. The process of claim 1, wherein the hydrocarbon conversion process is one or more of oxidative dehydrogenation, hydrogenation, transfer hydrogenation, aromatization, and reforming processes.

8. The process of claim 1, wherein the hydrocarbon conversion process is a dehydrogenation process.

9. The process of claim 1, wherein the catalytic composite comprises from about 0.01 weight percent to about 5.0 weight percent the first component, from about 0.01 weight percent to about 5.0 weight percent the second component, and from about 0.01 weight percent to about 5.0 weight percent the third component.

10. The process of claim 1, wherein the first component is platinum, the second component is potassium, and the third component is tin.
